(19) United States
(12) Patent Application Publication
(10) Pub. No.: US 2022/0298214 A1
(43) Pub. Date: Sep. 22, 2022

(54) Highly effective blood-glucose-lowering protein drug

(71) Applicant: Xintrum Pharmaceuticals, Ltd., Nanjing (CN)

(72) Inventors: Hai Chen, Nanjing (CN); Gaoyong Liao, Nanjing (CN); Yi Zhang, Nanjing (CN)

(21) Appl. No.: 17/827,604

(22) Filed: May 27, 2022

Related U.S. Application Data
(63) Continuation of application No. PCT/CN2021/131857, filed on Nov. 19, 2021.

(30) Foreign Application Priority Data
Dec. 10, 2020 (CN) 202011437565.8

(51) Int. Cl. C07K 14/605; A61P 3/10

(52) U.S. Cl. CPC C07K 14/605; A61P 3/10; A61K 38/00

(57) ABSTRACT

This invention provides a protein that is highly effective in lowering blood glucose. Experimental results have shown that the protein can be used to prepare a drug for treating diabetes.

### TECHNICAL FIELD

The present invention relates to the field of biotechnology and more particularly to a highly effective blood-glucose-lowering protein drug.

### DESCRIPTION OF RELATED ART

Glucagon-like peptide-1 (GLP-1) can lower the blood glucose level effectively by binding to the GLP-1 receptor in the human body. More specifically, GLP-1 acts directly on the pancreas to promote the release of insulin and inhibit the secretion of glucagon. Also, GLP-1 inhibits peristalsis of the stomach, delays gastric emptying, and acts on the central nervous system to suppress appetite. Currently there are eight GLP-1 receptor agonists (GLP-1RAs) on the market, and the most common safety issues with those GLP-1RAs are adverse reactions in the gastrointestinal tract. It has been reported that increasing the dose of a GLP-1RA slowly may limit the side effect, and enhance the therapeutic effect, of the GLP-1RA. However, not all the patients treated with GLP-1RAs have attained the goal of lowering their blood glucose level.

Now that the therapeutic effect of a GLP-1RA cannot be raised by increasing its dose, it is imperative to develop a drug that not only has the activity of GLP-1RA, but also can activate other nutrition and energy metabolism adjustment pathways that are associated with diabetes.

Glucose-dependent insulinotropic polypeptide (GIP) is the major cause of the postprandial incretin effect in normal people and functions differently from GLP-1. GIP plays an important role in the metabolism of adipose-tissue glucose and fat by regulating glucose consumption, lipolysis, and the activity of lipoprotein lipase.

There have been experimental reports stating that a higher level of insulin secretion can be achieved in a healthy subject by administering two drugs, or more specifically a GLP-1RA and a GIP agonist, at the same time than by administering the GLP-1RA alone.

Therefore, the design and obtainment of a single molecule that serves as a GLP-1/GIP double-receptor agonist and can bring about the blood glucose lowering effects of both GLP-1 and GIP is expected to result in more desirable blood glucose control.

### BRIEF SUMMARY OF THE INVENTION

One objective of the present invention is to provide a drug that has a significant blood glucose lowering effect.

According to the present invention, a protein having the amino acid sequence of the following general formula has agonist activity at two receptors, namely the GLP-1 receptor and the GIP receptor, at the same time.

The present invention provides a double-receptor (i.e., the GLP-1 receptor and the GIP receptor) agonist protein having the amino acid sequence of general formula I:

YGEGTFTSDYSIYLDKQA-a-FV-b-WLLA-c-GPSSGAPPPS general formula I.

In general formula I,
-a-represents AKE or QRA;
-b-represents N or E; and
-c-represents G or Q.

In other words, general formula I represents the following two amino acid sequences, in each of which the amino acids are sequentially arranged in the order from an N terminus to a C terminus:

YGEGTFTSDYSIYLDKQAAKEFVNWLLAGGPSSGAPPPS,
and
YGEGTFTSDYSIYLDKQAQRAFVEWLLAQGPSSGAPPPS.

According to the present invention, each of the two amino acid sequences represented by general formula I, i.e., SEQ ID NO: 1 and SEQ ID NO: 2, is connected at the C terminus with a polypeptide serving as a non-functional area for increasing protein stability, and the amino acid sequences of the resulting proteins are defined by SEQ ID NO: 3 and SEQ ID NO: 4 respectively.

Experimental results have shown that a protein having the amino acid sequence of SEQ ID NO: 4 (hereinafter referred to as GGF7 for short) as well as a protein having the amino acid sequence of SEQ ID NO: 3 (hereinafter referred to as GGF2 for short) has agonist activity at both the GLP-1 receptor and the GIP receptor.

The double-receptor agonist proteins provided by the present invention exist in the form of homodimers.

The present invention involves constructing a double-receptor agonist protein in an expression vector,

The expression vector is a eukaryotic expression vector and can be introduced into a host cell by transient transfection or stable transfection.

The host cell is a mammalian cell, and the mammalian cell is a Chinese hamster ovary (CHO) cell or a human embryonic kidney 293 cell.

More specifically, the following research work was conducted for the present invention:

1. Nucleotide sequences corresponding respectively to the amino acid sequences of GGF2 and GGF7 were designed and synthesized in accordance with the codon usage bias of the CHO cell and were each constructed in the pcDNA3.4 vector to produce the expression vectors pcDNA3.4-GGF2 and pcDNA3.4-GGF7.

2. Each of the expression vectors was transfected into a CHO cell through a transfection reagent, and after culturing, cell supernatants in which GGF2 and GGF7 were respectively expressed were obtained.

3. Each of GGF2 and GGF7 was separated and purified through Protein A and was tested by sodium dodecyl sulfate-polyacrylamide gel electrophoresis (SDS-PAGE).

4. The bioactivity of GGF2 and GGF7 was tested with the GLP-1 receptor and the GIP receptor.

5. A study on the activity of GGF2 and GGF7 in lowering blood glucose was conducted on db/db diabetic model mice.

The proteins provided by the present invention can activate the GLP-1 receptor and the GIP receptor at the same time (see embodiment 2 and 3), lower the non-fasting blood glucose (NFBG) level and the fasting blood glucose (FBG) level extremely significantly (P<0.0001) (see embodiment 4), and lower the glycated hemoglobin (HbA1c) level significantly (P<0.01) as compared with the commercially available Dulaglutide (abbreviated as Dul) (see embodiment 4), and are therefore more effective in lowering blood glucose than Dulaglutide.

### BRIEF DESCRIPTION OF THE SEVERAL VIEWS OF THE DRAWINGS

FIG. 1: The result of agarose gel electrophoresis of a positive clone of the constructed and polymerase chain reaction (PCR)-screened expression vector pcDNA3.4-GGF2 for the protein GGF2.

FIG. 2: The result of agarose gel electrophoresis of a positive clone of the constructed and PCR-screened expression vector pcDNA3.4-GGF7 for the protein GGF7.

FIG. 3: The result of non-reduced SDS-PAGE of supernatants in which the proteins GGF2 and GGF7 were respectively expressed.

FIG. 4: The result of non-reduced SDS-PAGE of Protein A-purified GGF2 and GGF7.

FIG. 5: The NFBG level lowering effect of the protein GGF2 on db/db diabetic model mice.

FIG. 6: The FBG level lowering effect of the protein GGF2 on db/db diabetic model mice.

FIG. 7: The NFBG level lowering effect of the protein GGF7 on db/db diabetic model mice.

FIG. 8: The FBG level lowering effect of the protein GGF7 on db/db diabetic model mice.

FIG. 9: The HbAa1c level lowering effects of the proteins GGF2 and GGF7 on db/db diabetic model mice.

### SEQUENCE LISTING INFORMATION

SEQ ID NO: 1: Amino acid sequence (1) represented by general formula I.

SEQ ID NO: 2: Amino acid sequence (2) represented by general formula I.

SEQ ID NO: 3: Amino acid sequence of the double-receptor agonist protein GGF2.

SEQ ID NO: 4: Amino acid sequence of the double-receptor agonist protein GGF7.

### DETAILED DESCRIPTION OF THE INVENTION

In the following embodiments, the preparation methods of the two double-receptor agonist proteins GGF2 and GGF7 are identical and therefore will be disclosed as one method. The same applies to the bioactivity determination methods of GGF2 and GGF7.

As mentioned in connection with the embodiments, GGF2 and GGF7 refer to two double-receptor agonist proteins prepared according to the present invention, and “Dul” refers to Dulaglutide, a commercially available blood glucose lowering drug made by Eli Lilly and Company and used in the control group.

### Embodiment 1: Preparation of the Double-Receptor Agonist Proteins GGF2 and GGF7

### 1. Construction of an Expression Vector for each of the Proteins GGF2 and GGF7

Based on the features of CHO cell expression, the amino acid sequence of each of GGF2 and GGF7 was optimized and reverse-translated into a nucleotide sequence, which was then synthesized chemically, digested with the restriction enzymes EcoRI and BamHI, and purified with a gel extraction kit to produce enzymatically digested DNA fragments. The pcDNA3.4 vector was digested with the same two restriction enzymes EcoRI and BamHI, and the enzymatic digestion product was purified with a DNA purification kit.

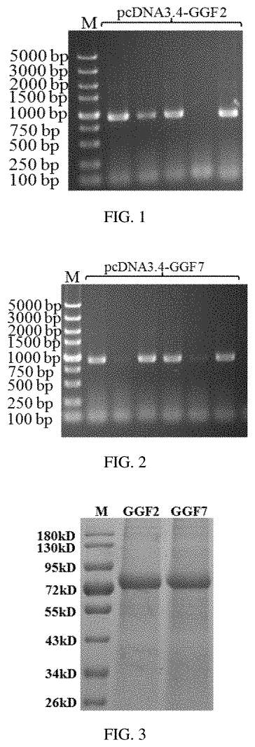

Using the T4 DNA ligase, each of the GGF2 and GGF7 genes that had been digested with EcoRI and BamHI was ligated to the pcDNA3.4 vector that had been digested with the same two enzymes, and the ligated insert and vector were chemically transformed into a Top10 competent cell. Each single colony that grew after the transformation was screened by PCR in order to obtain positive clones. The size of a PCR product in which a target gene had been successfully ligated to the expression vector was about 900-1000 bp, and FIG. 1 and FIG. 2 show the results of agarose gel electrophoresis of such PCR products.

Some of the PCR-screened positive clones pcDNA3.4-GGF2 and pcDNA3.4-GGF7 were selected for sequencing, and a comparison and analysis of the sequences obtained revealed that the nucleotide sequence of each of GGF2 and GGF7 was consistent with the corresponding theoretical sequence.

### 2. Expression of the proteins GGF2 and GGF7

The host cells (CHO cells) were cultured under the following conditions: A culture medium designed for CHO cell expression was used, the orbital shaker had an orbit diameter of 2.5 cm and a rotation speed of 120 rpm, the carbon dioxide concentration was 8%, and the temperature was 37° C. Subculturing was performed when the cell count of the CHO cells reached 4-6×10⁶ cells/mL, and the cell density in the subcultures was adjusted to 2-5×10⁵ cells/mL. The day before transfection, the cells were subcultured again, and the cell density was adjusted to 3-4×10⁵ cells/mL. Prior to transfection, the cell density was adjusted with a complete culture medium to 6×10⁶ cells/mL. An appropriate transfection volume was chosen according to the purpose of the experiment. In a 1.5-mL centrifuge tube, the expression vector pcDNA3.4-GGF2/pcDNA3.4-GGF7 in Step 1 was added, after being purified, at a ratio of 0.133 μg per 10⁶ to-be-transfected cells, and then optiPRO SFM was added until a final volume of 6.7 μL per 10⁶ to-be-transfected cells was reached. After that, the centrifuge tube was shaken at a moderate speed until its contents were thoroughly mixed. In another 1.5-mL centrifuge tube, two reagents, namely ExpiFectamine CHO Reagent (a transfection reagent) and optiPRO SFM, were added at a ratio of 0.533 μL per 10⁶ cells and a ratio of 6.134 μL per 10⁶ cells respectively, and the centrifuge tube was shaken at a moderate speed until its contents were thoroughly mixed Immediately after that, the diluted transfection reagent was mixed with the vector solution, and the mixture was placed at room temperature for 1-5 min. The vector-transfection reagent mixture solution was then added into the cell suspension by drops, and the resulting mixture was transferred at once to a 37° C., 8% CO₂ shaker for culturing. After 20 hours of culture, an enhancer and a feed were added into the shaker flask at a ratio of 1 μL per 10⁶ to-be-transfected cells and a ratio of 40 μL per 10⁶ to-be-transfected cells respectively, and the culture conditions were adjusted to 32° C. and 5% CO₂. On the 9th day of culture, the culture supernatant was collected by centrifugation and kept at a temperature not higher than −70° C. Samples were then taken for non-reduced SDS-PAGE, whose results are shown in FIG. 3, indicating that GGF2 and GGF7 were successfully expressed.

### 3. Separation and Purification of the Proteins GGF2 and GGF7

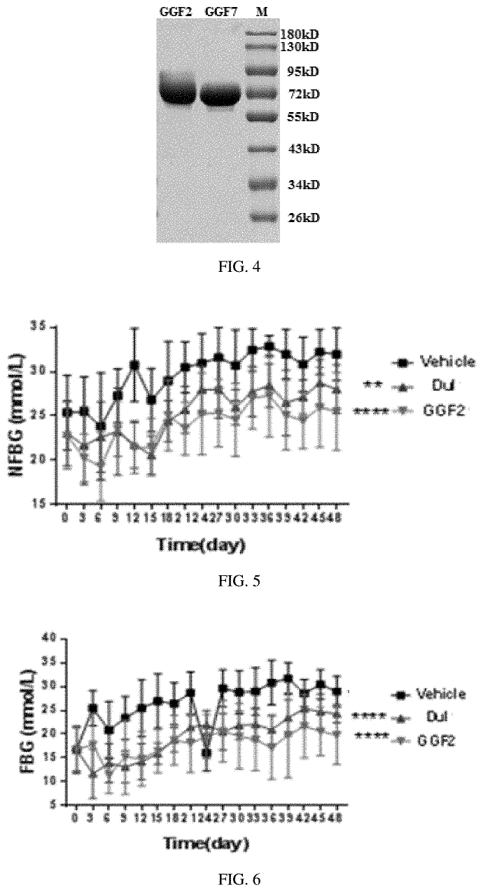

The GGF2/GGF7-expressed supernatant in Step 2 was separated and purified with the Protein A filler. Phosphate-buffered saline (PBS) was used as the equilibrium buffer, and a pH3.0, 100 mM citric acid/sodium citrate buffer as the elution buffer. The eluate corresponding to the elution peak was collected and substituted into the PBS buffer to obtain the protein GGF2/GGF7. The protein content was determined by the ultraviolet method. The results of non-reduced SDS-PAGE are shown in FIG. 4.

### Embodiment 2: Determination of the Binding Activity of the Double-Receptor Agonist Proteins (GGF2 and GGF7) at the GLP-1 Receptor

Experimental method and its principle:

The bioactivity of the double-receptor agonist proteins in activating the GLP-1 receptor was determined by the luciferase reporter gene method. The cell HEK293-GLP1R-CRE-Luc can express the GLP-1 receptor stably, with CRE specifically promoting expression of the luciferase, and this signal pathway can be specifically promoted by the cell HEK293-GLP1R-CRE-Luc binding to the GLP-1 receptor after being treated with either double-receptor agonist protein. As the last step, therefore, a substrate is added to produce a chemiluminescent signal, whose intensity is positively correlated to the bioactivity of the corresponding double-receptor agonist protein.

The steps of the method are as follows:

Poly-L-lysine was added to a 96-hole plate at a concentration of 0.1 mg/mL and in an amount of 100 μL per hole, and the hole walls were allowed to be coated with the poly-L-lysine at 37° C. for 24 h. Before use, the plate was washed once with sterile double-distilled water and then put into an incubator for the water to evaporate. After that, the 96-hole plate was inoculated with the cell HEK293-GLP1R-CRE-Luc at a cell density of 3×10⁴/hole, and the number of the holes to be inoculated was determined according to the number of the samples to be tested. Each drug was applied to 3 columns×9 rows of cells. Each of the proteins GGF2, GGF7, and Dul was diluted to the concentrations of 10, 2, 0.4, 0.08, 0.016, 0.0032, 0.00064, 0.000128, and 0 nM. The culture solution in each hole was discarded, before 100 μL of diluted GGF2/GGF7 solution was added to each hole. After 24 hours of culture, the 96-hole plate was taken out of the incubator, and 100 μL of One-Glo Luciferase Assay solution, which was prepared in advance and had been brought to room temperature, was added to each hole, thoroughly mixed, and allowed to rest for 5 min in order for lysis to take place. The numerical values of each hole were determined with a multifunctional microplate reader by the chemiluminescence method. The chemiluminescence value-concentration curve of each fusion protein was fitted with the software GraphPad Prism (using the three-parameter nonlinear regression equation fitting mode). The EC₅₀ values, which can be used to indicate the bioactivity of GGF2 and GGF7, were subsequently calculated.

The experimental results are shown in Table 1.

TABLE 1
Affinity of GGF2 and GGF7 toward the GLP-1 receptor

| Double-receptor agonist protein | GGF2 | GGF7 | Dul |
|---|---|---|---|
| EC₅₀ value (pM) | 24.34 | 46.06 | 41.17 |

Conclusion of the experiment: Both GGF2 and GGF7 were able to bind to the GLP-1 receptor. Compared with the positive-control-group drug Dul, GGF2 produced a significant effect whereas GGF7 produced a similar effect.

### Embodiment 3: Determination of the Binding Activity of the Double-Receptor Agonist Proteins (GGF2 and GGF7) at the GIP Receptor

Experimental method and its principle:

The bioactivity of the double-receptor agonist proteins in activating the GIP receptor was determined with CHO-K1-GIPR, which is a cell strain capable of over-expression of the GIP receptor, and the underlying principle is as follows. When each double-receptor agonist protein has reacted with the cell CHO-K1-GIPR for a while, the cyclic adenosine monophosphate (cAMP) content of the cell will have increased, and the cAMP content is positively correlated to the activity of the drug within a certain range. Therefore, the bioactivity of the drug/agonist to be tested can be known by measuring how the cAMP signal in the cell varies with the concentration of the drug. In this experiment, the bioactivity of each double-receptor agonist protein in activating the GIP receptor was determined by a method entailing competition between externally marked cAMP and the cAMP produced.

The steps of the method are as follows:

Well-grown CHO-K1-GIPR cells were selected, digested with pancreatin, washed twice with Dulbecco's phosphate-buffered saline (DPBS), resuspended with a lx stimulation buffer, and counted. The cell density was then adjusted to 6×10⁵ cells/mL. Each hole of a 96-hole plate was added with 5 μL of the cell suspension, followed by 5 μL of the to-be-tested drug GGF2 or GGF7 or the control-group Dul at a corresponding concentration, which started with the highest concentration of 200 nM and was sequentially reduced by 5 times gradient dilution. Also, a cAMP standard area was established, in which each hole was added with 5 μL of cAMP at a corresponding concentration. The 96-hole plate was covered with a sealing film and placed in an incubator for incubation at 37° C. for 30min. Following that, the 96-hole plate was taken out of the incubator, and each hole was added with 5 μL of a cAMP working fluid. After a thorough mix, each hole was further added with 5 μL of an anti-cAMP-cryptate working fluid. After another thorough mix, the plate was covered with a sealing film again, allowing incubation to take place at room temperature for 1 hour. The ratio values of (signal 665 nm/signal 620 nm)*10000 were read from a multifunctional microplate reader, and data processing and analysis was carried out with the software GraphPad Prism6. The EC50 values of the drugs under test, i.e., GGF2 and GGF7, were subsequently calculated.

The experimental results are shown in Table 2.

TABLE 2
Affinity of GGF2 and GGF7 toward the GIP receptor

| Double-receptor agonist protein | GGF2 | GGF7 | Dul |
|---|---|---|---|
| EC₅₀ value (pM) | 177.9 | 35.3 | No signal |

Conclusion of the experiment: Both GGF2 and GGF7 were able to bind to the GIP receptor, but GGF7 had the higher affinity toward the receptor. Dulaglutide (Dul) and the GIP receptor produced no binding signal.

### Embodiment 4: Investigation on the Activity of the Double-Receptor Agonist Proteins in Lowering the Blood Glucose of db/db Diabetic Model Mice

Purpose of the experiment:

To investigate the blood glucose lowering activity of the double-receptor agonist proteins GGF2 and GGF7 in db/db type-2 diabetic model mice.

Method of the experiment:

32 db/db male mice were chosen for the experiment. The mice were 6 weeks old when received and underwent adaptive feeding in an animal house for 1 week. After the week, blood was drawn from the tail tip for an NFBG test. After fasting for 6 hours, blood was drawn from the tail tip again for an FBG test, and 50 μL of blood was drawn from the retrobulbar venous plexus and subjected to whole blood separation in order to perform an HbA1c test on the plasma obtained. The experimental animals were randomly divided into four groups according primarily to the FBG level and secondarily to body weight. The four groups were the model group (i.e., the vehicle group, with n=8), the positive drug group (i.e., the Dul group, with n=8), the GGF2 administration group (i.e., the GGF2 group, with n=8), and the GGF7 administration group (i.e., the GGF7 group, with n=8). Every three days, the four groups were given their respective drugs/placebo through subcutaneous injection over the neck at 16.67 nmol/kg. Prior to drug administration, blood was drawn from the tail tip in order to perform an NFBG test. After fasting for 6 hours, an FBG test was conducted. The drugs were administered continuously for 16 times. On the 3rd day after the last drug administration, the mice were fasted for 6 hours before their FBG and HbA1c were tested.

Experimental results:

FIG. 5 and FIG. 6 show the NFBG level and FBG level lowering effects of the protein GGF2 on db/db diabetic model mice;

FIG. 7 and FIG. 8 show the NFBG level and FBG level lowering effects of the protein GGF7 on db/db diabetic model mice; and

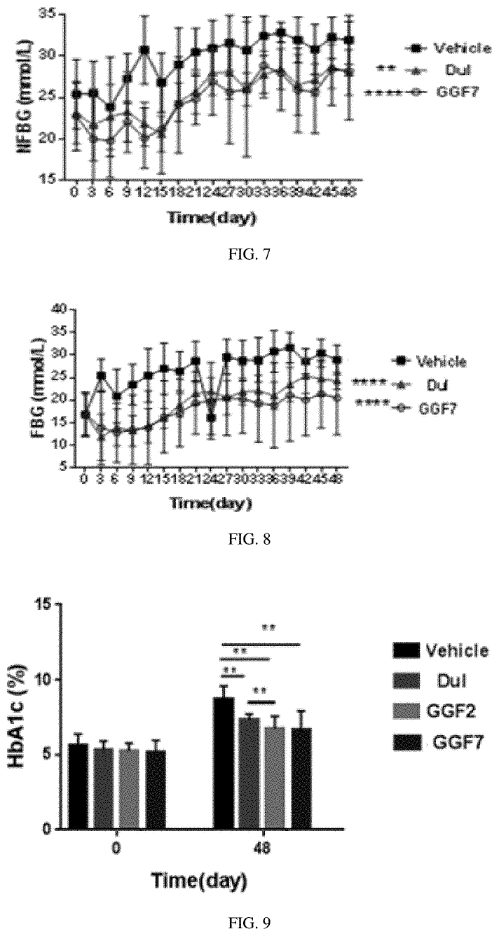

FIG. 9 shows the HbA1c level lowering effects of the proteins GGF2 and GGF7 on db/db diabetic model mice.

The following can be known from the results shown in the drawings:

Compared with the vehicle group, both GGF2 and GGF7:

1) lowered the NFBG level extremely significantly (****, P<0.0001);

2) lowered the FBG level extremely significantly (****, P<0.0001); and

3) lowered the HbA1c level significantly (**, P<0.01).

Compared with the positive drug (Dulaglutide, or Dul) group:

1) both GGF2 and GGF7 lowered the HbA1c level significantly (**, P<0.01) and more effectively than Dulaglutide (Dul) ;

2) both GGF2 and GGF7 lowered the NFBG level more effectively than Dulaglutide (Dul); and

3) both GGF2 and GGF7 lowered the FBG level more effectively than Dulaglutide (Dul). Conclusion of the experiment:

The animal experiment shows that the double-receptor agonist proteins GGF2 and GGF7 of the present invention have a blood glucose lowering function.

1. A protein comprising the amino acid sequence of general formula I:
YGEGTFTSDYSIYLDKQA-a-FV-b-WLLA-c-GPSSGAPPPS general formula I
wherein the general formula I comprises amino acids sequentially arranged in an order from an N terminus to a C terminus, and the letters a, b, and c represent the following amino acids respectively:
a represents AKE or QRA;
b represents N or E; and
c represents G or Q.

2. The protein of claim 1 comprising the amino acid sequence of SEQ ID NO: 1 or SEQ ID NO: 2.

3. A protein comprising the amino acid sequence of SEQ ID NO:1 or SEQ ID NO:2.

4. A protein comprising the amino acid sequence of SEQ ID NO:3 or SEQ ID NO:4.

5. A pharmaceutical composition for treating diabetes, comprising a protein comprising the amino acid sequence of SEQ ID NO:3 or SEQ ID NO:4 as an active ingredient.

6. A blood glucose lowering agent comprising the protein of claim 3.

7. A blood glucose lowering agent comprising the protein of claim 4.

8. A glucagon-like peptide 1 (GLP-1) receptor agonist comprising the protein of claim 1.

9. A glucose-dependent insulinotropic polypeptide (GIP) receptor agonist comprising the protein of claim 1.


SEQUENCE LISTING (4 sequences)

SEQ ID NO: 1 - protein, 39 aa, Artificial Sequence
  Synthetic
YGEGTFTSDYSIYLDKQAAKEFVNWLLAGGPSSGAPPPS

SEQ ID NO: 2 - protein, 39 aa, Artificial Sequence
  Synthetic
YGEGTFTSDYSIYLDKQAQRAFVEWLLAQGPSSGAPPPS

SEQ ID NO: 3 - protein, 283 aa, Artificial Sequence
  Synthetic
YGEGTFTSDYSIYLDKQAAKEFVNWLLAGGPSSGAPPPSGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 283 aa, Artificial Sequence
  Synthetic
YGEGTFTSDYSIYLDKQAQRAFVEWLLAQGPSSGAPPPSGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG